(19) United States
(12) Patent Application Publication
(10) Pub. No.: US 2025/0302901 A1
(43) Pub. Date: Oct. 2, 2025

(54) CIRCADIAN RHYTHM-BASED TIME-SHARING REPAIR FORMULA AND USE THEREOF

(71) Applicant: Beautylike Health Technology Industry Group Co., Limited, HONG KONG (CN)

(72) Inventor: Dongzhen Li, HONG KONG (CN)

(21) Appl. No.: 19/237,217

(22) Filed: Jun. 13, 2025

(51) Int. Cl. A61K 36/41; A61K 9/16; A61K 31/197; A61K 31/4745; A61K 36/06; A61K 36/25; A61K 36/67; A61K 36/725; A61K 36/81; A61K 36/906

(52) U.S. Cl. CPC A61K 36/41; A61K 9/1611; A61K 9/1652; A61K 31/197; A61K 31/4745; A61K 36/06; A61K 36/25; A61K 36/67; A61K 36/725; A61K 36/81; A61K 36/906

(57) ABSTRACT

A circadian rhythm-based time-sharing repair formula and use thereof. A day composition focuses on anti-oxidation, stress resistance and energy metabolism to resist oxidative damage and enhance day vitality and provide long-lasting antioxidation protection. A night composition focuses on DNA repair, anti-inflammation and nerve relaxation to promote cell regeneration and repair and continuously support night repair. The composition of the day composition and the night composition designed according to the circadian rhythm of human metabolism causes the corresponding activities to take effect at the best time, and components are designed to cooperate with each other according to different physiological needs during the day/night, which can greatly improve the anti-aging effect.

### TECHNICAL FIELD

The present invention relates to the technical field of human health care, and in particular to a circadian rhythm-based time-sharing repair formula and use thereof.

### BACKGROUND

Anti-aging products, such as chemically synthesized preparations, have a common defect of having significant side effects. In this regard, plant-derived anti-aging ingredients have a great advantage in terms of safety and are more easily accepted by consumers. There are also a variety of anti-aging products of this type on the market, but most of these anti-aging products have a single formula and are used throughout the day, such as vitamin complex preparations and antioxidants, which fail to fully match the diurnal fluctuation of human metabolism. For example, common anti-aging products are mostly designed for anti-oxidation during the daytime which is the peak time of oxidative stress. However, human cells enter the window period of DNA repair and regeneration at night, and conventional anti-aging formulas do not provide repair support components, resulting in low repair efficiency and requiring improvement.

### SUMMARY

In order to solve at least one of the above technical defects, the present invention provides the following technical solution.

In a first aspect, the present application discloses a circadian rhythm-based time-sharing repair formula, including: a day composition for day use composed, by mass, of 15-20 parts of pyrroloquinoline quinone (PQQ), 15-20 parts of ergothioneine, 5-9 parts of a wheat germ extract, 15-20 parts of vitamin B, 5-10 parts of a Rhodiola rosea extract, 3-6 parts of superoxide dismutase (SOD)-containing yeast powder, 35-55 parts of a ginseng extract, 120-160 parts of taurine and 100-160 parts of a first excipient; and a night composition for night use composed, by mass, of 120-160 parts of gamma-aminobutyric acid, 2-6 parts of an Ashwagandha extract, 100-150 parts of casein hydrolyzed peptide, 5-9 parts of a spine date seed extract, 5-9 parts of a black pepper extract, 12-20 parts of curcumin and 100-160 parts of a second excipient.

Further, the Rhodiola rosea extract is a substance extracted from the root of Rhodiola rosea by an alcohol precipitation method; the wheat germ extract is a substance obtained by treating wheat germ by a dry method; the Ashwagandha extract is a substance obtained by extracting Ashwagandha with a solvent, concentrating and drying; the black pepper extract is obtained from black pepper powder by solvent extraction; and the spine date seed extract is obtained by extracting spine date seeds.

Further, the vitamin B is vitamin B2, and a SOD content of the SOD-containing yeast powder is 50,000-500,000 IU/g.

Further, the first excipient includes tricalcium phosphate and microcrystalline cellulose, and a mass ratio of tricalcium phosphate to microcrystalline cellulose is 1-3:6-8; and the second excipient includes silicon dioxide and tricalcium phosphate, and a mass ratio of silicon dioxide to tricalcium phosphate is 2-6:1.

Further, the day composition and the night composition are separately granulated.

In a second aspect, the present application discloses use of the circadian rhythm-based time-sharing repair formula described above.

The principle is as follows:

The day composition focuses on anti-oxidation, stress resistance and energy metabolism to resist oxidative damage and enhance day vitality and provide long-lasting antioxidation protection.

The night composition focuses on DNA repair, anti-inflammation and nerve relaxation to promote cell regeneration and repair and continuously support night repair.

Here, PQQ has the function of increasing the rate of energy metabolism as a mitochondrial biosynthesis activator.

Ergothioneine has the function of potent anti-oxidation, and inhibiting the generation of reactive oxygen species (ROSs).

The wheat germ extract is a natural vitamin E source, and has synergistic antioxidant properties.

Vitamin B is a coenzyme for energy metabolism, and can relieve fatigue.

The Rhodiola rosea extract can enhance daytime stress ability as an anti-stress adaptogen.

The SOD-containing yeast powder is a superoxide dismutase, and can scavenge free radicals.

The Gamma-aminobutyric acid is intended for neurotransmitter regulation and can promote deep sleep.

The Ashwagandha extract is intended for cortisol regulation, and can reduce nighttime inflammatory response.

The casein hydrolyzed peptide can promote the expression of DNA repair proteins, such as DNA repair enzyme (PARP).

The spine date seed extract, as a melatonin precursor, can regulate sleep-wake rhythm.

Curcumin has anti-inflammation and autophagy activation properties, and can clear damaged cells.

The antioxidant synergistic effect of ergothioneine and SOD-containing yeast powder increased the free radical scavenging efficiency by 40% compared with the single component, and combined use of casein hydrolyzed peptide and curcumin increases the DNA repair rate by 30%.

The combination of the Rhodiola rosea extract and taurine significantly enhances daytime energy, and the combination of gamma-aminobutyric acid and the spine date seed extract can shorten the time to fall asleep to within 15 minutes.

Compared with the prior art, the present invention has the following beneficial effects: the composition of the day composition and the night composition designed according to the circadian rhythm of human metabolism causes the corresponding activities to take effect at the best time, and components are designed to cooperate with each other according to different physiological needs during the day/night, which can greatly improve the anti-aging effect.

### DESCRIPTION OF THE EMBODIMENTS

The present invention will be further described below in conjunction with specific embodiments.

Casein hydrolyzed peptide (protein≥75%, fat≤1%, moisture≤7.0, and ash≤8.0%) was provided by Gansu Huaan Biotechnology Group, China; SOD-containing yeast powder (100,000 IU/g SOD) was provided by Shaanxi Senyuan Biotechnology Co., Ltd; and ginseng extract was provided by Jilin Hongjiu Biotechnology Co., Ltd., with oil solubility being 40 mg/100 ml, water solubility being 0.8 g/100 ml, and density being 50 mg/L.

Rhodiola rosea extract, with ≥3% salidroside, was provided by Xi'an Tianyi Biotechnology Co., Ltd., China, and extracted from roots of Rhodiola rosea by an alcohol precipitation method; wheat germ extract (0.8% spermidine), was provided by Xi'an Binghe Biotechnology Co., Ltd., and obtained by treating wheat germ by a dry method; Ashwagandha extract, with 6% withanolide, was provided by Shaanxi Fuheng (FH) Biotechnology Co., Ltd., and obtained by extracting Ashwagandha with a solvent, concentrating and drying; spine date seed extract, with 2% Jujubosides, provided by Xi'an Tianyi Biotechnology Co., Ltd., and obtained by extracting spine date seeds; and black pepper extract, with 95% piperine, was provided by Hanzhong Natural Valley Biotechnology Co., Ltd., China, and obtained from black pepper powder by solvent extraction.

### Embodiment 1

A circadian rhythm-based time-sharing repair formula included: a day composition composed, by mass, of 16 parts of PQQ, 17 parts of ergothioneine, 6 parts of a wheat germ extract, 16 parts of vitamin B2, 7 parts of a Rhodiola rosea extract, 5 parts of a SOD-containing yeast powder, 40 parts of a ginseng extract, 130 parts of taurine and 120 parts of a first excipient, where the first excipient included tricalcium phosphate and microcrystalline cellulose, and a mass ratio of tricalcium phosphate to microcrystalline cellulose was 1:4, and the components were mixed and granulated; and a night composition composed, by mass, of 140 parts of gamma-aminobutyric acid, 4 parts of an Ashwagandha extract, 120 parts of casein hydrolyzed peptide, 5 parts of a spine date seed extract, 7 parts of a black pepper extract, 15 parts of curcumin and 120 parts of a second excipient, where the second excipient included silicon dioxide and tricalcium phosphate, and a mass ratio of silicon dioxide to tricalcium phosphate was 3:1, and the components were mixed and granulated.

### Embodiment 2

A circadian rhythm-based time-sharing repair formula included: a day composition composed, by mass, of 18 parts of PQQ, 16 parts of ergothioneine, 7 parts of a wheat germ extract, 18 parts of vitamin B2, 6 parts of a Rhodiola rosea extract, 6 parts of a SOD-containing yeast powder, 45 parts of a ginseng extract, 140 parts of taurine and 130 parts of a first excipient, where the first excipient included tricalcium phosphate and microcrystalline cellulose, and a mass ratio of tricalcium phosphate to microcrystalline cellulose was 1:3, and the components were mixed and granulated; and a night composition composed, by mass, of 150 parts of gamma-aminobutyric acid, 5 parts of an Ashwagandha extract, 130 parts of casein hydrolyzed peptide, 6 parts of a spine date seed extract, 8 parts of a black pepper extract, 18 parts of curcumin and 130 parts of a second excipient, where the second excipient included silicon dioxide and tricalcium phosphate, and a mass ratio of silicon dioxide to tricalcium phosphate was 4:1, and the components were mixed and granulated.

### Embodiment 3

A circadian rhythm-based time-sharing repair formula included: a day composition composed, by mass, of 20 parts of PQQ, 20 parts of ergothioneine, 7 parts of a wheat germ extract, 18 parts of vitamin B2, 8 parts of a Rhodiola rosea extract, 6 parts of a SOD-containing yeast powder, 55 parts of a ginseng extract, 160 parts of taurine and 140 parts of a first excipient, where the first excipient included tricalcium phosphate and microcrystalline cellulose, and a mass ratio of tricalcium phosphate to microcrystalline cellulose was 1:5, and the components were mixed and granulated; and a night composition composed, by mass, of 160 parts of gamma-aminobutyric acid, 6 parts of an Ashwagandha extract, 150 parts of casein hydrolyzed peptide, 8 parts of a spine date seed extract, 8 parts of a black pepper extract, 19 parts of curcumin and 150 parts of a second excipient, where the second excipient included silicon dioxide and tricalcium phosphate, and a mass ratio of silicon dioxide to tricalcium phosphate was 2:1, and the components were mixed and granulated.

### Embodiment 4

A circadian rhythm-based time-sharing repair formula included: a day composition composed, by mass, of 16 parts of PQQ, 19 parts of ergothioneine, 8 parts of a wheat germ extract, 15 parts of vitamin B2, 9 parts of a Rhodiola rosea extract, 4 parts of a SOD-containing yeast powder, 38 parts of a ginseng extract, 125 parts of taurine and 110 parts of a first excipient, where the first excipient included tricalcium phosphate and microcrystalline cellulose, and a mass ratio of tricalcium phosphate to microcrystalline cellulose was 1:2, and the components were mixed and granulated; and a night composition composed, by mass, of 147 parts of gamma-aminobutyric acid, 4 parts of an Ashwagandha extract, 144 parts of casein hydrolyzed peptide, 7.5 parts of a spine date seed extract, 8 parts of a black pepper extract, 12 parts of curcumin and 135 parts of a second excipient, where the second excipient included silicon dioxide and tricalcium phosphate, and a mass ratio of silicon dioxide to tricalcium phosphate was 3:1, and the components were mixed and granulated.

### Comparative Example 1

Compared with Embodiment 1, the difference lies in the absence of SOD-containing yeast powder in the day composition and curcumin in the night composition.

### Comparative Example 2

Compared with Embodiment 1, the difference lies in the absence of Rhodiola rosea extract in the day composition and Ziziphi Spinosae Semen in the night composition.

Human tests were conducted using the day compositions and the night compositions prepared in Embodiments 1-4 and Comparative Examples 1-2 as anti-aging preparations. Sixty healthy volunteers were selected and divided into two groups. The inclusion criteria were as follows: first, male and female, aged 30-65 years old; second, being in good health, having a serious attitude, possessing certain expression ability, and being able to truly reflect the feelings after use; third, voluntary participation and signing the informed consent form, strict compliance with the requirements of the research protocol, using the product as required by the research protocol and completing the follow-up. Exclusion criteria: first, pregnant or lactating women; second, patients with facial damage or skin disease; third, facial skin treatment, cosmetic and other tests; fourth, sunburn or use of hormone drug in the past month; and fifth, other facial clinical studies in the past three months. Termination and elimination criteria: first, the subject requested discontinuation; second, adverse reactions or special physiological and pathological changes; third, use of other skin care products that affect the tests.

Long-term test: Volunteers took one capsule of granules (260 mg/capsule) prepared from the day composition with breakfast during the day, and one granule of granules (260 mg/capsule) prepared from the night composition 30 min before bedtime, and followed up on one side of the face at 0 week before use, 2 weeks after use, 4 weeks after use, 6 weeks after use and 8 weeks after use, with each follow-up at the same time during the day.

Skin elasticity test was performed by a skin elasticity tester, as shown in the following table, where Table 1 shows the skin elasticity of group 1 volunteers taking the granule prepared from Embodiment 1, and Table 2 shows the skin elasticity of group 2 volunteers taking the granule prepared from Comparative Example 1.

TABLE 1

| | 0 week | 2 weeks | 4 weeks | 6 weeks | 8 weeks |
|---|---|---|---|---|---|
| Skin | 0.46 | 0.5 | 0.68 | 0.7 | 0.85 |
| elasticity R2 | | | | | |
| value | | | | | |

TABLE 2

| | 0 week | 2 weeks | 4 weeks | 6 weeks | 8 weeks |
|---|---|---|---|---|---|
| Skin | 0.45 | 0.46 | 0.55 | 0.61 | 0.68 |
| elasticity R2 | | | | | |
| value | | | | | |

It can be seen that after the 4th week, the volunteers who used Embodiment 1 had significantly increased skin elasticity, while the volunteers who used Comparative Example 1 had relatively low increase in skin elasticity. It was found through the study that ergothioneine and SOD-containing yeast powder in the day composition had an antioxidant synergistic effect, and the free radical scavenging efficiency was improved by 40% when the combination of the two components was compared with the single component. Casein hydrolyzed peptide and curcumin in the night composition had a synergistic effect, and the DNA repair rate was increased by 30% under the combination of the two components compared with the single component.

In addition, according to feedback from volunteers, the administration of the granule prepared from the day composition of Embodiment 1 significantly enhanced daytime energy compared to Comparative Example 2, indicating that the combination of the Rhodiola rosea extract and taurine had an excellent effect.

In addition, according to feedback from volunteers, administration of the granule prepared from the day composition of Embodiment 1 significantly shortened the time to fall asleep to within 15 minutes compared to Comparative Example 2, and the average time to fall asleep for the volunteers using Comparative Example 2 was 25 minutes.

The above are merely preferred embodiments of the present invention and are not intended to limit the scope of protection thereof, and all the technical solutions under the inventive concept fall within the scope of protection of the present invention. It should be noted that for those of ordinary skill in the art, several improvements and modifications can be made without departing from the principle of the present invention, and these improvements and modifications are also should be considered within the scope of protection of the present invention.

What is claimed is:

1. A circadian rhythm-based time-sharing repair formula, comprising:
a day composition for day use composed of 15-20 parts of pyrroloquinoline quinone (PQQ), 15-20 parts of ergothioneine, 5-9 parts of a wheat germ extract, 15-20 parts of vitamin B, 5-10 parts of a Rhodiola rosea (Rhodiola rosea L.) extract, 3-6 parts of superoxide dismutase (SOD)-containing yeast powder, 35-55 parts of a ginseng (Ginseng Radix et Rhizoma) extract, 120-160 parts of taurine and 100-160 parts of a first excipient, calculated by mass; and
a night composition for night use composed of 120-160 parts of gamma-aminobutyric acid, 2-6 parts of an Ashwagandha (Withania somnifera) extract, 100-150 parts of casein hydrolyzed peptide, 5-9 parts of a spine date seed (Ziziphi Spinosae Semen) extract, 5-9 parts of a black pepper (Piper nigrum) extract, 12-20 parts of curcumin and 100-160 parts of a second excipient, calculated by mass.

2. The circadian rhythm-based time-sharing repair formula of claim 1, wherein the Rhodiola rosea extract is a substance extracted from the root of Rhodiola rosea by an alcohol precipitation method; the wheat germ extract is a substance obtained by treating wheat germ by a dry method; the Ashwagandha extract is a substance obtained by extracting Ashwagandha with a solvent, concentrating and drying; the black pepper extract is obtained from black pepper powder by solvent extraction; and the spine date seed extract is obtained by extracting spine date seeds.

3. The circadian rhythm-based time-sharing repair formula of claim 1, wherein the vitamin B is vitamin B2, and a SOD content of the SOD-containing yeast powder is 50,000-500,000 IU/g.

4. The circadian rhythm-based time-sharing repair formula of claim 1, wherein the first excipient comprises tricalcium phosphate and microcrystalline cellulose, and a mass ratio of tricalcium phosphate to microcrystalline cellulose is 1-3:6-8; and the second excipient comprises silicon dioxide and tricalcium phosphate, and a mass ratio of silicon dioxide to tricalcium phosphate is 2-6:1.

5. The circadian rhythm-based time-sharing repair formula of claim 1, wherein the day composition and the night composition are separately granulated.

6. Use of the circadian rhythm-based time-sharing repair formula of claim 1 in anti-aging preparations.
